(19) United States
(12) Patent Application Publication
(10) Pub. No.: US 2024/0156703 A1
(43) Pub. Date: May 16, 2024

(54) COMPOSITIONS AND METHODS FOR TREATING KERATIN FIBERS

(71) Applicant: L 'Oreal, Paris (FR)

(72) Inventors: Kazumitsu KAWAKAMI, Westfield, NJ (US); Liliana XAVIER, Elizabeth, NJ (US)

(21) Appl. No.: 17/977,126

(22) Filed: Oct. 31, 2022

(51) Int. Cl. A61K 8/41; A61Q 5/12

(52) U.S. Cl. CPC A61K 8/416; A61Q 5/12; A61K 2800/34

(57) ABSTRACT

The disclosure relates to compositions for treating, caring for, and/or conditioning keratin fibers, such as hair, comprising at least two amine compounds, including at least one quaternary alkyl amine and at least one tertiary alkyl amine; at least two nonionic surfactants including at least one nonionic surfactant with an HLB value of greater than or equal to about 15 and at least one nonionic surfactant with an HLB value of less than or equal to about 5; at least one branched alkyl alcohol; and at least one solvent. The disclosure also relates to methods of using the compositions.

### TECHNICAL FIELD

The present disclosure relates to compositions for treating, caring for, and/or conditioning keratin fibers, such as hair, and methods of using the compositions.

### BACKGROUND

Consumers generally desire to have hair that looks and feels healthy. A variety of factors can lead to an undesirable appearance and/or the feel of hair, and can lead to lank body and decreased volume, such as natural greasiness, sweat, skin cells from the scalp, pollution, and dirt. In addition, environmental factors can affect the appearance or feel of the hair. This can be seen, for example, in regions with hard water. This is because hard water leaves mineral deposits, which accumulate over time, on the hair and eventually prevents moisture intake into the hair. The hair becomes dry, frizzy, and is prone to tangles. Other environmental factors, such as strong sun, wind, cold air, extreme temperature variations and changes in air humidity can also damage the hair. The static and dry winter air contributes to moisture loss. Abrupt change from cold outdoor air to warm indoor air can cause the cuticle layers of the hair to lose moisture quickly into the atmosphere.

Further, processes to alter the color or shape of the hair, including those using harsh chemicals such as bleaching, dyeing, straightening, and permanent waving, the use of heat, styling tools, brushes, etc., can damage the hair fiber. Such damage may appear as split ends, broken hair, flyaway hair, etc., and may cause the hair to feel rough and look dull and unhealthy.

Hair care products are used to address these concerns. Conventional cleansing compositions such as shampoos, for example, which contain surfactants such as anionic, nonionic, and/or amphoteric type surfactants, can be employed to remove the diverse types of soils typically present on the hair. These compositions, while providing good cleansing power, may yield poor cosmetic properties due to the fact that the nature of such a cleansing treatment may result in a less conditioned or rough feel of the hair due to, for example, the gradual removal of the natural or applied fats, lipids, or proteins contained in or at the surface of the hair.

Thus, a hair treatment or conditioner composition can be used on hair in order to confer conditioning, smoothing, and/or softening properties to the hair. However, certain components in conventional hair treatment and/or conditioner compositions may cause build-up on the hair, may cause the hair to feel heavy or weighed-down, and/or may not provide sufficient care or conditioning benefits.

Formulating treatment or conditioning compositions in order to avoid these drawbacks, while still providing effective care and/or conditioning benefits, has proven to be challenging, in part because components that are known to provide conditioning benefits are typically those that are also responsible for the properties that are considered undesirable, for example leaving a greasy or weighed-down feeling or appearance of the hair. Additionally, formulating such compositions presents challenges in achieving not only the desired benefits for the treated keratin fibers, but also desirable and necessary properties of the product, for example viscosity and stability.

Thus, the present disclosure relates to compositions comprising a combination of components that work together synergistically to address the above problems, providing excellent care and conditioning benefits while also providing the hair with excellent cosmetic properties.

### SUMMARY

The disclosure relates to compositions for treating, caring for, and/or conditioning keratin fibers, particularly hair, and methods of using the compositions. In various embodiments, the compositions are rinse-off or leave-in compositions. The compositions provide, in various embodiments, excellent care and conditioning properties, such as ease of detangling and/or softness, while also providing beneficial cosmetic properties, such as volume, smoothness, and/or a lightweight feeling, to keratin fibers, particularly hair.

In various embodiments, the disclosure relates to compositions for treating, caring for, and/or conditioning hair, comprising at least two amine compounds, including at least one quaternary alkyl amine and at least one tertiary alkyl amine; at least two nonionic surfactants including at least one nonionic surfactant with an HLB value of greater than or equal to about 15 and at least one nonionic surfactant with an HLB value of less than or equal to about 5; at least one branched alkyl alcohol; and at least one solvent.

In some embodiments, the composition comprises at least one quaternary alkyl amine chosen from quaternary ammonium salts of formula (I) below. For example, the at least one quaternary alkyl amine may be chosen from behentrimonium chloride, cetrimonium chloride, behentrimonium methosulfate, or mixtures thereof. The total amount of quaternary alkyl amine compounds present in the composition may, in some embodiments, range from about 0.5% to about 10%, such as from about 1% to about 8%, from about 1.5% to about 6%, or from about 2% to about 5% by weight, based on the total weight of the composition.

In some embodiments, the composition comprises at least one tertiary alkyl amine chosen from tertiary amido amine compounds. For example, the composition may comprise stearamidopropyldimethylamine. The total amount of tertiary alkyl amine compounds may, in some embodiments, range from about 0.01% to about 5%, such as from about 0.1% to about 3%, from about 0.2% to about 2%, or from about 0.3% to about 1% by weight, based on the total weight of the composition.

In some embodiments, it may be advantageous to have total amounts of quaternary and tertiary alkyl amine compounds present in the hair treatment composition such that a weight ratio of the total amount of quaternary alkyl amines to the total amount of tertiary alkyl amines is greater than about 1, for example ranges from about 1 to about 12, from about 2 to about 11, from about 3 to about 10, from about 4 to about 9, from about 5 to about 8, from about 6 to about 7.5, or from about 7 to about 7.5.

In some embodiments, the nonionic surfactants with an HLB value of greater than or equal to about 15 may comprise Polysorbate-80, Ceteth-20, Ceteth-24, Laureth-21, Laureth-23, Steareth-20, Steareth-21, or the like. In various embodiments, the total amount of nonionic surfactants with an HLB value of greater than or equal to about 15 in the composition ranges from about 0.01% to about 5%, from about 0.1% to about 4%, from about 0.2% to about 3%, from about 0.3% to about 2%, or from about 0.5% to about 1.5% by weight, based on the total weight of the composition.

In some embodiments, the nonionic surfactants with an HLB value of lower than or equal to about 5 may comprise glyceryl stearate, glyceryl laurate, glycol stearate, sorbitan isostearate, sorbitan oleate, sorbitan sesquioleate, sorbitan stearate, sorbitan trioleate, or the like. In various embodiments, the total amount of nonionic surfactants with an HLB value of lower than or equal to about 5 in the composition ranges from about 0.01% to about 5%, such as from about 0.01% to about 3%, from about 0.05% to about 2%, from about 0.075% to about 1.5%, or from about 0.1% to about 1% by weight, based on the total weight of the composition.

In some embodiments, it may be advantageous to include the high HLB and low HLB nonionic surfactants in the composition in amounts so that a weight ratio of the total amount of nonionic surfactants with an HLB value of greater than or equal to about 15 to the total amount of nonionic surfactants with an HLB value of lower than or equal to about 5 is greater than about 1, for example ranges from about 1 to about 10, such as from about 2 to about 9, from about 3 to about 8, from about 3 to about 7, from about 4 to about 7, from about 4 to about 6, or from about 4 to about 5.

In some embodiments, branched alkyl alcohols may be chosen from those containing at least 8 carbon atoms, such as from 12 to 40 carbon atoms or from 16 to 24 carbon atoms, such as, for example, butyloctanol, hexyloctanol, butyldecanol, hexyldecanol, octyldodecanol, hexyldecanol, ethylhexanol, or mixtures thereof. In various embodiments, the composition comprises a total amount of branched alkyl alcohols ranging from about 0.01% to about 6%, such as from about 0.1% to about 4%, from about 0.2% to about 3%, or from about 0.5% to about 1.5% by weight, based on the total weight of the composition.

The compositions comprise at least one solvent, which can include water and/or one or more non-aqueous solvents. In various embodiments, the compositions comprise additional components, such as, for example, cationic polymers, carboxylic acids, thickening agents, or combinations thereof. In some embodiments, the compositions are free or substantially free of silicones. The pH of the compositions is typically less than about 7, for example ranging from about 2.5 to about 6, or from about 3 to about 5.

In some embodiments, methods of using the compositions according to the disclosure comprise applying a rinse-off composition to keratin fibers, e.g. hair, which may be wet, damp, or dry, optionally leaving the composition in the hair for a brief resting period, such as about one minute, about two minutes, etc., and rinsing the composition from the hair.

In some embodiments, methods of using the compositions according to the disclosure comprise applying a leave-in composition to keratin fibers, e.g. hair, which may be wet, damp, or dry, and leaving the composition in the hair until the hair is washed next in the ordinary course of personal grooming, such as, for example about 24 hours or about 48 hours.

### DETAILED DESCRIPTION

The disclosure relates to compositions for treating, caring for, and/or conditioning keratin fibers, e.g. hair, and to methods of using the compositions. The compositions comprise a combination of components that work together to surprisingly balance excellent care and conditioning benefits with excellent cosmetic properties of the hair. The methods of using the compositions comprise applying the compositions to the hair in order to treat, care for, and/or condition the hair.

I. Compositions

Compositions according to the disclosure comprise at least two amine compounds, including at least one quaternary alkyl amine and at least one tertiary alkyl amine; at least two nonionic surfactants including at least one nonionic surfactant with an HLB value of greater than or equal to about 15 and at least one nonionic surfactant with an HLB value of less than or equal to about 5; at least one branched alkyl alcohol; and at least one solvent.

### Amine Compounds

Compositions according to the disclosure comprise at least two amine compounds, including at least one quaternary alkyl amine and at least one tertiary alkyl amine. Salts of amine compounds can also be used, and are expressly included in the term “amine compound” unless stated otherwise. In some embodiments, the amine compounds are selected from the group consisting of quaternary alkyl amines and tertiary alkyl amines.

The amine compounds may, in various embodiments, be chosen from alkyl amines, for example mono-long alkyl amines. Mono-long alkyl amines may, in various embodiments, comprise at least 8 carbon atoms in the alkyl chain, such as from 12 to 30 carbon atoms, from 16 to 24 carbon atoms, or from 18 to 22 carbon atoms. Amido amines, such as alkyl amino amines can also be chosen.

In various embodiments, the compositions comprise a total amount of amine compounds ranging from about 0.1% to about 20%, for example from about 0.5% to about 18%, from about 0.75% to about 15%, from about 1% to about 12%, from about 1.5% to about 10%, from about 2% to about 8%, from about 2.5% to about 6%, or from about 3% to about 5% by weight, relative to the total weight of the composition. In other embodiments, the compositions comprise a total amount of quaternary and tertiary alkyl amines ranging from about 0.5% to about 20%, for example from about 0.5% to about 18%, from about 0.75% to about 15%, from about 1% to about 12%, from about 1.5% to about 10%, from about 2% to about 8%, from about 2.5% to about 6%, or from about 3% to about 5% by weight, relative to the total weight of the composition.

In at least some embodiments, it may be advantageous to include amounts of quaternary and tertiary alkyl amines in the composition in order to achieve a weight ratio of the total amount of quaternary alkyl amine(s) to the total amount of tertiary alkyl amine(s) that is greater than 1, for example greater than about 2, greater than about 3, greater than about 4, greater than about 5, greater than about 6, greater than about 7, greater than about 8, greater than about 9, or greater than about 10, such as, for example, ranging from about 1 to about 12, from about 2 to about 11, from about 3 to about 10, from about 4 to about 9, from about 5 to about 8, from about 6 to about 7.5, or from about 7 to about 7.5.

### Quaternary Alkyl Amines

Compositions according to the disclosure include at least one quaternary alkyl amine. Optionally, the compositions may comprise at least two, at least three, at least four, etc., quaternary alkyl amines.

Exemplary and non-limiting quaternary alkyl amines that can be used include those of the general formula (I) below:

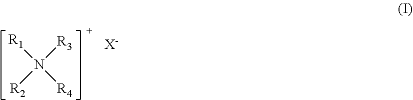

wherein R₁, R₂, R₃ and R₄ are independently chosen from saturated or unsaturated, linear or branched, aliphatic hydrocarbon radicals comprising from 1 to about 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms, and X⁻ is an anion, for example a halide, e.g., chloride, bromide, or iodide, a (C1-C4) alkyl sulfate, a (C1-C4) alkylsulfonate, or a (C1-C4) alkylarylsulfonate, a methanesulfonate, a phosphate, a nitrate, a tosylate, an anion derived from an organic acid such as an acetate or a lactate, or any other anion that is compatible with the ammonium bearing an ester function. In some embodiments, the anion X⁻ is bromide, chloride, or methosulfate. Optionally, at least one of R₁, R₂, R₃ and R₄ has at least 8 carbon atoms, such as from 12 to 30 carbon atoms, from 16 to 24 carbon atoms, or from 18 to 22 carbon atoms.

In various embodiments, the quaternary alkyl amines may be chosen from monoalkyl quaternary alkyl amines, dialkyl quaternary alkyl amines, or mixtures thereof. Mention may be made, for example, of salts of tetraalkylammonium compounds, such as the bromide, chloride, or methosulfate salts. For example, alkyltrimethylammonium compounds in which the alkyl group comprises at least 8 carbon atoms, such as from 12 to 22 carbon atoms or from 14 to 20 carbon atoms, may be chosen. As a further example, dialkyldimethylammonium compounds in which at least one alkyl group comprises at least 8 carbon atoms, such as from 12 to 22 carbon atoms or from 14 to 20 carbon atoms, may be chosen. By way of non-limiting example, behenyltrimethylammonium bromide, chloride, and/or methosulfate, distearyldimethylammonium bromide, chloride, and/or methosulfate, cetyltrimethylammonium bromide, chloride, and/or methosulfate, dodecyltrimethylammonium bromide, chloride, and/or methosulfate, or benzyldimethylstearylammonium bromide, chloride, and/or methosulfate may be chosen. In various embodiments, the quaternary amine comprises, consists essentially of, or consists of behenyltrimethylammonium chloride (behentrimonium chloride), cetyltrimethylammonium chloride (cetrimonium chloride), or a mixture thereof.

As further non-limiting examples, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, behenalkonium chloride, benzethonium chloride, cetylpyridinium chloride, lauralkonium chloride, cetalkonium chloride, or cethylamine hydrofluoride, may be chosen.

Combinations of two or more of any of the foregoing, such as three or more, four or more, etc., may also be chosen.

In various embodiments, the total amount of quaternary alkyl amines may range up to about 15%, such as from about 0.5% to about 12%, from about 1% to about 10%, from about 1.5% to about 8%, or from about 2% to about 5% by weight, relative to the total weight of the composition. In certain embodiments, the total amount of quaternary alkyl amines ranges from about 0.5% to about 15%, such as from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 2% to about 15%, from about 2% to about 12%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 15%, from about 2.5% to about 12%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 3% to about 15%, from about 3% to about 12%, from about 3% to about 10%, from about 3% to about 9%, from about 3% to about 8%, from about 3% to about 7%, from about 3% to about 6%, from about 3% to about 5%, from about 3% to about 4%, from about 3.5% to about 15%, from about 3.5% to about 12%, from about 3.5% to about 10%, from about 3.5% to about 9%, from about 3.5% to about 8%, from about 3.5% to about 7%, from about 3.5% to about 6%, from about 3.5% to about 5%, or from about 3.5% to about 4% by weight, relative to the total weight of the composition. In further embodiments, the composition may comprise a total amount of quaternary alkyl amines ranging from about 1.5% to about 5.5%, from about 1.5% to about 5%, from about 1.5% to about 4.5%, from about 1.5% to about 4%, from about 1.5% to about 3.75%, from about 2% to about 5.5%, from about 2% to about 4.5%, from about 2% to about 3.75%, from about 2.5% to about 5.5%, from about 2.5% to about 5%, from about 2.5% to about 4.5%, from about 2.5% to about 4%, from about 2.5% to about 3.75%, from about 2.75% to about 5.5%, from about 2.75% to about 5%, from about 2.75% to about 4.5%, from about 2.75% to about 4%, from about 2.75% to about 3.75%, from about 3% to about 5.5%, from about 3% to about 4.5%, from about 3% to about 3.75%, from about 3.25% to about 5.5%, from about 3.25% to about 5%, from about 3.25% to about 4.5%, from about 3.25% to about 4%, or from about 3.25% to about 3.75% by weight, relative to the total weight of the composition.

### Tertiary Alkyl Amines

Compositions according to the disclosure comprise at least one tertiary alkyl amine. Optionally, the compositions may comprise at least two, at least three, at least four, etc., tertiary alkyl amines.

Exemplary and non-limiting tertiary alkyl amines that can be used include those of the general formula (II) below:

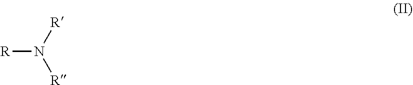

wherein R, R′ and R″ are independently chosen from saturated or unsaturated, linear or branched, aliphatic hydrocarbon radicals comprising from 1 to about 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms. Optionally, at least one of R, R′ and R″ has at least 8 carbon atoms, such as from 12 to 30 carbon atoms, from 16 to 24 carbon atoms, or from 18 to 22 carbon atoms.

In some embodiments of formula (II), R′ and/or R″ are chosen from linear alkyl chains comprising from 1 to 6, such as from 1 to 4, or from 1 to 3 carbon atoms, and R is chosen from an alkoxy, alkoxycarbonylalkyl, polyoxyalkylene, alkylamido, alkylamidoalkyl, hydroxyalkyl, aromatic, aryl, or alkylaryl radical comprising at least 8 carbon atoms, such as from 12 to 30 carbon atoms, from 16 to 24 carbon atoms, or from 18 to 22 carbon atoms.

Further exemplary and non-limiting tertiary alkyl amines that can be used include those of the general formula (III) below:

R₄—A—R₅—B (III)
wherein R₄ is a saturated or unsaturated, linear or branched alkyl chain with 8 to 24 carbon atoms, R₅ is a linear or branched alkyl chain with 1 to 4 carbon atoms, A is:
and B is:

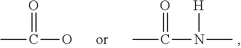

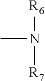

wherein R₆ and R₇, which may be the same or different, are chosen from an alkyl chain with 1 to 4 carbon atoms, a hydroxyl alkyl chain with 1 to 4 carbon atoms, or a di-hydroxyl alkyl chain with 2 to 4 carbon atoms.

By way of non-limiting example, tertiary amido amines having an alkyl group of from about 12 to about 22 carbons can be chosen. Non-limiting examples of amido amines that can be used include brassicamidopropyl dimethylamine, stearamidopropyldimethylamine, stearamidopropyldiethylamine, stearamidoethyl-diethylamine, stearamidoethyldimethylamine, palmitamidopropyldimethylamine, palmitamidopropyldiethylamine, palmitamidoethyldiethylamine, palmitamidoethyl-dimethylamine, behenamidopropyl-dimethylamine, behenamidopropyldiethylamine, behenamidoethyldiethylamine, behenamidoethyldimethylamine, arachidamidopropyl-dimethylamine, arachidamido-propyldiethylamine, arachidamidoethyldiethylamine, or arachidamidoethyldimethylamine, diethylaminoethylstearamide.

In at least some embodiments, the tertiary amine comprises, consists essentially of, or consists of tertiary amido amines. In some embodiments, the tertiary amine comprises, consists essentially of, or consists of stearamidopropyldimethylamine.

In various embodiments, the total amount of tertiary alkyl amines may range up to about 5%, such as from about 0.01% to about 5%, from about 0.1% to about 4%, from about 0.2% to about 3%, or from about 0.3% to about 2% by weight, relative to the total weight of the composition. In certain embodiments, the total amount of tertiary alkyl amines may range from about 0.1% to about 5%, about 0.1% to about 4%, about 0.1% to about 3%, about 0.1% to about 2%, about 0.1% to about 1%, about 0.2% to about 5%, about 0.2% to about 4%, about 0.2% to about 3%, about 0.2% to about 2%, about 0.2% to about 1%, about 0.3% to about 5%, about 0.3% to about 4%, about 0.3% to about 3%, about 0.3% to about 2%, about 0.3% to about 1%, about 0.4% to about 5%, about 0.4% to about 4%, about 0.4% to about 3%, about 0.4% to about 2%, about 0.4% to about 1%, about 0.5% to about 5%, about 0.5% to about 4%, about 0.5% to about 3%, about 0.5% to about 2%, or from about 0.5% to about 1% by weight, based on the total weight of the composition. In some embodiments, the composition comprises a total amount of tertiary alkyl amines ranging from about 0.15% to about 0.85%, from about 0.2% to about 0.8%, from about 0.25% to about 0.75%, from about 0.3% to about 0.7%, from about 0.35% to about 0.65%, or from about 0.4% to about 0.6% by weight, relative to the total weight of the composition.

### Nonionic Surfactants

Compositions according to the disclosure comprise at least two nonionic surfactants, including at least one nonionic surfactant with an HLB value of greater than or equal to about 15 (referred to in some instances herein as a “high HLB nonionic surfactant”) and at least one nonionic surfactant with an HLB value of less than or equal to about 5 (referred to in some instances herein as a “low HLB nonionic surfactant”). Optionally, the compositions may comprise at least two, at least three, at least four, etc., high HLB nonionic surfactants, and/or at least two, at least three, at least four, etc., low HLB nonionic surfactants.

The HLB value refers to the hydrophilic-lipophilic balance of the surfactant, and can be measured experimentally or calculated. In the present application, the HLB values are the values at about 25° C.

The HLB value of the nonionic surfactants can, for example, be determined experimentally according to the book by Puisieux and Seiller, “Galenica 5: Les systemes disperses [Galenics 5: Dispersed systems]—Volume I—Agents de surface et emulsions [Surface agents and emulsions]—Chapter IV—Notions de HLB et de HLB critique [Notions of HLB and of critical HLB], pages 153-194—paragraph 1.1.2. Détermination de HLB par voie expérimentale [Experimental determination of HLB],” pages 164-180.

The HLB values of nonionic surfactants can also be calculated by the following formula:

HLB=(20)(MW_{H})/(MW_{H})(MW_{L})
where MW_{H} is the molar weight of the hydrophilic portion and MW_{L} is the molar weight of the lipophilic portion. See Griffin, W. C. Calculation of HLB values of Nonionic Surfactants, J. Soc. Cos. Chem. (1954), 5, 249-256.

Compositions according to the disclosure can include any nonionic surfactant, but the compositions include at least one high HLB nonionic surfactant and at least one low HLB nonionic surfactant. Non-limiting examples of useful high and/or low nonionic surfactants include polyoxyethylene glyceryl fatty acid esters, sorbitan fatty acid esters, polyoxyethylene sorbitan fatty acid esters, polyoxyethylene alkyl ethers, alkylated polysaccharides, alkyl glycosides, sucrose fatty acid esters, polyglycol fatty acid esters, alkaloyl diethanolamides, and fatty acid isopropanolamides. By way of example only, in at least some embodiments, ethoxylated or propoxylated alcohols and alkyl phenols, which, in various embodiments, contain a straight or branched C₈₋₂₂ alkyl group, for example C₁₀₋₂₀ or C₁₂₋₂₀, and from about 6 to about 30, such as from about 6 to about 25, ethylene oxide or propylene oxide groups (high HLB nonionic surfactants) or from about 1 to about 5 ethylene oxide or propylene oxide groups (low HLB nonionic surfactants), may be chosen. In other embodiments, esters of acids and alcohols, e.g. glyceryl esters of fatty acids, may be chosen.

In at least some embodiments, the nonionic surfactants present in the compositions are selected from the group consisting of high HLB nonionic surfactants and low HLB nonionic surfactants.

By way of example only, high HLB nonionic surfactants may be chosen from cetyl alcohol polyoxyethylenated with 20 EO (Ceteth-20) (HLB 15.7); cetyl alcohol polyoxyethylenated with 24 EO (Ceteth-24) (HLB 16.3); lauryl alcohol polyoxyethylenated with 21 EO (Laureth-21) (HLB 15.5); lauryl alcohol polyoxyethylenated with 23 EO (Laureth-23) (HLB 16.3); stearyl alcohol polyoxyethylenated with 20 EO (Steareth-20) (HLB 15.2); stearyl alcohol polyoxyethylenated with 21 EO (Steareth-21) (HLB 15.5); polyoxyethylene (20) sorbitan monolaurate (Polysorbate-20) (HLB 16.7); and polyoxyethylene (6) oleyl ether (Oleth-20) (HLB 15.3). In some embodiments, the high HLB nonionic surfactant comprises, consists essentially of, or consists of polyoxyethylene (20) sorbitan monolaurate (Polysorbate-20).

By way of example only, low HLB nonionic surfactants may be chosen from stearyl alcohol polyoxyethylenated with 2 EO (Steareth-2) (HLB 4.9); glyceryl laurate (HLB 5.1); glyceryl stearate (HLB 3.8); glycol stearate (HLB 2.9); sorbitan isostearate (HLB 4.7); sorbitan oleate (HLB 4.3); sorbitan sesquioleate (HLB 3.7); sorbitan stearate (HLB 4.7); sorbitan trioleate (HLB 1.8); and 2-[2-[(Z)-octadec-9-enoxy]ethoxy]ethanol (Oleth-2) (HLB 4.9). In some embodiments, the low HLB nonionic surfactant comprises, consists essentially of, or consists of glyceryl stearate, sorbitan oleate, or mixtures thereof.

In some embodiments, the high HLB nonionic surfactants are chosen from those having an HLB greater than or equal to 15, such as greater than 15, or greater than or equal to 16, such as greater than 16. In some embodiments, the low HLB nonionic surfactant are chosen from those having an HLB less than or equal to 5, such as less than 5, or less than or equal to 4, such as less than 4.

In various embodiments, the total combined amount of high HLB and low HLB nonionic surfactants may range from about 0.01% to about 5%, such as, for example, from about 0.1% to about 5%, from about 0.1% to about 4%, from about 0.2% to about 3%, from about 0.3% to about 2%, or from about 0.5% to about 1.5% by weight, relative to the total weight of the composition. In certain embodiments, the total amount of high HLB and low HLB nonionic surfactants may range from about 0.1% to about 5%, about 0.1% to about 4%, about 0.1% to about 3%, about 0.1% to about 2%, about 0.1% to about 1%, about 0.2% to about 5%, about 0.2% to about 4%, about 0.2% to about 3%, about 0.2% to about 2%, about 0.2% to about 1%, about 0.3% to about 5%, about 0.3% to about 4%, about 0.3% to about 3%, about 0.3% to about 2%, about 0.3% to about 1%, about 0.4% to about 5%, about 0.4% to about 4%, about 0.4% to about 3%, about 0.4% to about 2%, about 0.4% to about 1%, about 0.5% to about 5%, about 0.5% to about 4%, about 0.5% to about 3%, about 0.5% to about 2%, or from about 0.5% to about 1% by weight, based on the total weight of the composition. In some embodiments, the composition comprises a total combined amount of high HLB and low HLB nonionic surfactants ranging from about 0.1% to about 1.5%, from about 0.2% to about 1.4%, from about 0.3% to about 1.3%, from about 0.4% to about 1.2%, from about 0.5% to about 1.1%, or from about 0.5% to about 1% by weight, relative to the total weight of the composition.

In some embodiments, the total amount of high HLB nonionic surfactants present in the composition may range from about 0.01% to about 5%, such as, for example, from about 0.05% to about 5%, from about 0.1% to about 4%, from about 0.2% to about 3%, from about 0.3% to about 2%, or from about 0.5% to about 1.5% by weight, relative to the total weight of the composition. In certain embodiments, the total amount of high HLB nonionic surfactants may range from about 0.1% to about 5%, about 0.1% to about 4%, about 0.1% to about 3%, about 0.1% to about 2%, about 0.1% to about 1%, about 0.2% to about 5%, about 0.2% to about 4%, about 0.2% to about 3%, about 0.2% to about 2%, about 0.2% to about 1%, about 0.3% to about 5%, about 0.3% to about 4%, about 0.3% to about 3%, about 0.3% to about 2%, about 0.3% to about 1%, about 0.4% to about 5%, about 0.4% to about 4%, about 0.4% to about 3%, about 0.4% to about 2%, about 0.4% to about 1%, about 0.5% to about 5%, about 0.5% to about 4%, about 0.5% to about 3%, about 0.5% to about 2%, or from about 0.5% to about 1% by weight, based on the total weight of the composition. In some embodiments, the composition comprises a total amount of high HLB nonionic surfactants ranging from about 0.1% to about 1.5%, from about 0.2% to about 1.4%, from about 0.3% to about 1.3%, from about 0.4% to about 1.2%, from about 0.5% to about 1.1%, or from about 0.5% to about 1% by weight, relative to the total weight of the composition.

In some embodiments, the total amount of low HLB nonionic surfactants present in the composition may range from about 0.01% to about 5%, such as from about 0.01% to about 3%, from about 0.05% to about 2%, from about 0.075% to about 1.5%, or from about 0.1% to about 1% by weight, based on the total weight of the composition. In certain embodiments, the total amount of low HLB nonionic surfactants may range from about 0.01% to about 5%, about 0.01% to about 4%, about 0.01% to about 3%, about 0.01% to about 2%, about 0.01% to about 1%, about 0.01% to about 0.5%, about 0.01% to about 0.25%, about 0.05% to about 5%, about 0.05% to about 4%, about 0.05% to about 3%, about 0.05% to about 2%, about 0.05% to about 1%, about 0.05% to about 0.5%, about 0.05% to about 0.25%, about 0.1% to about 5%, about 0.1% to about 4%, about 0.1% to about 3%, about 0.1% to about 2%, about 0.1% to about 1%, about 0.1% to about 0.5%, or about 0.1% to about 0.25% by weight, based on the total weight of the composition. In some embodiments, the composition comprises a total amount of low HLB nonionic surfactants ranging from about 0.05% to about 0.9%, from about 0.05% to about 0.8%, from about 0.05% to about 0.7%, from about 0.05% to about 0.6%, from about 0.05% to about 0.5%, from about 0.075% to about 0.9%, from about 0.075% to about 0.8%, from about 0.075% to about 0.7%, from about 0.075% to about 0.6%, from about 0.075% to about 0.5%, from about 0.1% to about 0.9%, from about 0.1% to about 0.8%, from about 0.1% to about 0.7%, from about 0.1% to about 0.6%, or from about 0.1% to about 0.5% by weight, relative to the total weight of the composition.

In at least some embodiments, it may be advantageous to include amounts of high HLB nonionic surfactants and low HLB nonionic surfactants in the composition in order to achieve a weight ratio in the composition of high HLB nonionic surfactant(s) to low HLB nonionic surfactant(s) of greater than about 1, for example greater than about 2, greater than about 3, greater than about 4, greater than about 5, greater than about 6, greater than about 7, or greater than about 8, such as, for example, ranging from about 1 to about 10, from about 2 to about 9, from about 3 to about 8, from about 3 to about 7, from about 4 to about 7, from about 4 to about 6, from about 4 to about 5, or from about 4 to about 4.5.

### Branched Alkyl Alcohols

Compositions according to the disclosure comprise at least one branched alkyl alcohol. Optionally, the compositions may comprise at least two, at least three, at least four, etc., branched alkyl alcohols.

In various exemplary embodiments, the alcohols may contain C₆-C₄₄ branched alkyl chains. In some embodiments, the alcohols may be chosen from branched, saturated alcohols containing at least 8 carbon atoms, for example from 12 to 40 carbon atoms or from 16 to 24 carbon atoms.

For example, in various embodiments the branched alkyl alcohols may be chosen from those of formulae (IV) or (V):

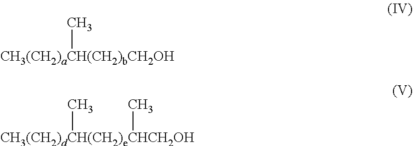

where a, b, d, and e are integers, with the sum of a and b ranging from 3 to 36, for example from 6 to 24 or from 12 to 20, and the sum of d and e ranging from 4 to 36, for example from 8 to 14.

By way of non-limiting example, butyloctanol, hexyloctanol, butyldecanol, hexyldecanol, octyldodecanol, hexyldecanol, ethylhexanol, 2-methyldodecanol, 2-methyltridecanol, 3-methyl pentadecanol, 4-methyl pentadecanol, 5-methyl pentadecanol, 6-methyl pentadecanol, 7-methyl pentadecanol, 8-methyl pentadecanol, 9-methyl pentadecanol, 10-methyl pentadecanol, 11-methyl pentadecanol, 12-methyl pentadecanol, 13-methyl pentadecanol, 3-methyl hexadecanol, 4-methyl hexadecanol, 5-methyl hexadecanol, 6-methyl hexadecanol, 7-methyl hexadecanol, 8-methyl hexadecanol, 9-methyl hexadecanol, 10-methyl hexadecanol, 11-methyl hexadecanol, 12-methyl hexadecanol, 13-methyl hexadecanol, 14-methyl hexadecanol, 2,3-methyl tetradecanol, 2,4-methyl tetradecanol, 2,5-methyl tetradecanol, 2,6-methyl tetradecanol, 2,7-methyl tetradecanol, 2,8-methyl tetradecanol, 2,9-methyl tetradecanol, 2,10-methyl tetradecanol, 2,11-methyl tetradecanol, 2,12-methyl tetradecanol, 2,3-methyl pentadecanol, 2,4-methyl pentadecanol, 2,5-methyl pentadecanol, 2,6-methyl pentadecanol, 2,7-methyl pentadecanol, 2,8-methyl pentadecanol, 2,9-methyl pentadecanol, 2,10-methyl pentadecanol, 2,11-methyl pentadecanol, 2,12-methyl pentadecanol, 2,13-methyl pentadecanol, 2-propylheptanol, 2-butyloctanol, 2-pentylnonanol, 2-hexyldecanol, 2-heptylundecanol, 2-octyldodecanol, 2-nonyltridecanol, 2-decyltetradecanol, 2-undecylpentadecanol, 2-dodecylhexadecanol, 2-tridecylheptadecanol, 2-tetradecyl octadecanol, 2-pentadecyl nonadecanol, 2-hexadecyleicosanol, 2-heptadecylheneicosanol, or mixtures of two or more thereof may be chosen. In some embodiments, the branched alkyl alcohol comprises, consists essentially of, or consists of octyldodecanol.

In various embodiments, the total amount of branched alkyl alcohols present in the composition may range from about 0.01% to about 6%, such as, for example, from about 0.05% to about 5%, from about 0.1% to about 4%, from about 0.2% to about 3%, from about 0.3% to about 2%, or from about 0.5% to about 1.5% by weight, relative to the total weight of the composition. In certain embodiments, the total amount of branched alkyl alcohols may range from about 0.1% to about 5%, about 0.1% to about 4%, about 0.1% to about 3%, about 0.1% to about 2%, about 0.1% to about 1.5%, about 0.2% to about 5%, about 0.2% to about 4%, about 0.2% to about 3%, about 0.2% to about 2%, about 0.2% to about 1.5%, about 0.3% to about 5%, about 0.3% to about 4%, about 0.3% to about 3%, about 0.3% to about 2%, about 0.3% to about 1.5%, about 0.4% to about 5%, about 0.4% to about 4%, about 0.4% to about 3%, about 0.4% to about 2%, about 0.4% to about 1.5%, about 0.5% to about 5%, about 0.5% to about 4%, about 0.5% to about 3%, about 0.5% to about 2%, or from about 0.5% to about 1.5% by weight, based on the total weight of the composition. In some embodiments, the composition comprises a total amount of branched alkyl alcohols ranging from about 0.1% to about 2.5%, from about 0.2% to about 2.25%, from about 0.3% to about 2%, from about 0.4% to about 1.75%, from about 0.5% to about 1.5%, or from about 0.75% to about 1.25% by weight, relative to the total weight of the composition.

### Solvent(s)

The compositions according to the disclosure comprise at least one solvent, optionally comprising water and/or at least one non-aqueous solvent. In various embodiments, the compositions comprise water and the total amount of water ranges from about 50% to about 95%, based on the total weight of the composition. In further embodiments, the total amount of water is from about 60% to about 95%, about 65% to about 95%, about 70% to about 95%, about 75% to about 95%, about 80% to about 95%, about 60% to about 90%, about 65% to about 90%, about 70% to about 90%, about 75% to about 90%, or about 80% to about 90% by weight, based on the total weight of the composition.

In some embodiments, the solvent includes one or more non-aqueous solvents, for example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of non-aqueous solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether, or mixtures of two or more thereof.

### Additional Optional Components

Compositions according to the disclosure may optionally, in various embodiments, comprise one or more additional components.

### Additional Surfactants

In addition to the high HLB and low HLB nonionic surfactants described above, it is also possible to include additional surfactants in compositions according to the disclosure. For example, the compositions may comprise one or more cationic surfactants other than the amine compounds described above, one or more anionic surfactants, and/or one or more nonionic surfactants other than high HLB and low HLB nonionic surfactants described above.

In at least some embodiments, the composition comprises at least one cationic surfactant. Exemplary and non-limiting optional cationic surfactants include cationic cellulose-based compounds, cationic starch-based compounds, cationic galactomannan compounds, and cationic silicone compounds, as well as mixtures of two or more thereof.

Exemplary and non-limiting optional anionic surfactants include acyl isethionates, acyl taurates, acyl sarcosinates, alkyl sulfonates, alkyl sulfosuccinates, alkyl ether sulfosuccinates, alkyl sulfoacetates, alkoxylated monoacids, and salts thereof, as well as mixtures of two or more thereof. In at least some embodiments, the composition is free or substantially free of anionic surfactants.

If present, the total amount of additional surfactants may vary, but may be, for example, at least about 0.1%, such as at least about 0.25%, at least about 0.5%, at least about 0.75%, or at least about 1% by weight, based on the total weight of the composition. In some embodiments, the total amount of additional surfactants may range from about 0.5% to about 5% by weight, relative to the total weight of the composition.

### Additional Fatty Compounds

Optionally, compositions according to the disclosure may comprise one or more fatty compounds other than branched alkyl alcohols. By way of non-limiting example, the compositions may optionally comprise oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), optionally branched and/or unsaturated fatty acids (e.g. myristic acid, palmitic acid, stearic acid, behenic acid, oleic acid, linoleic acid, linolenic acid, or isostearic acid), fatty alcohols other than those described above (e.g. cetearyl alcohol, cetanol, stearyl alcohol, oleyl alcohol, cetyl alcohol), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C₅-C₉ esters, trimethylolpropane polyesters, propylene glycol polyesters, or polyesters of hydrogenated castor oil), perfluorinated and/or organofluorinated oils, volatile or non-volatile silicone oils, fluorosilicone oils, or mixtures of two or more thereof. In at least one embodiment, the composition is free or substantially free of silicone oils.

If present, the total amount of additional fatty compounds may range 0.1% to about 15%, such as from about 0.5% to about 12%, from about 1% to about 10%, from about 1.5% to about 8%, or from about 2% to about 5% by weight, relative to the total weight of the composition. For example, the total amount of additional fatty compounds may range from about 1% to about 15%, from about 1% to about 12%, from about 1% to about 7%, from about 1% to about 5%, from about 1% to about 4%, from about 1% to about 3%, from about 1.5% to about 15%, from about 1.5% to about 12%, from about 1.5% to about 10%, from about 1.5% to about 7%, from about 1.5% to about 5%, from about 1.5% to about 4%, from about 1.5% to about 3%, from about 2% to about 15%, from about 2% to about 12%, from about 2% to about 10%, from about 2% to about 7%, from about 2% to about 5%, from about 2% to about 4%, from about 2% to about 3%, from about 2.5% to about 15%, from about 2.5% to about 12%, from about 2.5% to about 10%, from about 2.5% to about 7%, from about 2.5% to about 5%, from about 2.5% to about 4%, or from about 2.5% to about 3.5% by weight, relative to the total weight of the composition.

### Cationic Conditioning Polymers

Optionally, compositions according to the disclosure may comprise one or more cationic conditioning polymers that may provide additional conditioning benefits to the treated keratin fibers. By way of example only, poly(methacryloyloxyethyl trimethylammonium chloride), polyquaternium-37, quaternized cellulose derivatives, polyquaternium-4, polyquaternium-6, polyquaternium-10, cationic alkyl polyglycosides, cationized honey, cationic guar derivatives, polymeric dimethyl diallyl ammonium salts and copolymers thereof with esters and amides of acrylic acid and methacrylic acid, copolymers of vinyl pyrrolidone with quaternized derivatives of dialkylaminoalkyl acrylate and methacrylate, vinyl pyrrolidone-vinyl imidazolium methochloride copolymers, quaternized polyvinyl alcohol, polyquaternium-2, polyquaternium-7, polyquaternium-17, polyquaternium-18, polyquaternium-24, polyquaternium-27, or mixtures of two or more thereof may be chosen.

If present, the total amount of cationic conditioning polymers may vary, but may be, for example, at least about 0.1%, such as at least about 0.25%, at least about 0.5%, at least about 0.75%, or at least about 1% by weight, based on the total weight of the composition. In some embodiments, the total amount of cationic conditioning polymers may range from about 0.5% to about 5%, from about 0.5% to about 4%, from about 0.5% to about 3%, from about 0.5% to about 2%, from about 0.5% to about 1.5%, from about 0.6% to about 5%, from about 0.6% to about 4%, from about 0.6% to about 3%, from about 0.6% to about 2%, from about 0.6% to about 1.5%, from about 0.7% to about 5%, from about 0.7% to about 4%, from about 0.7% to about 3%, from about 0.7% to about 2%, from about 0.7% to about 1.5%, from about 0.8% to about 5%, from about 0.8% to about 4%, from about 0.8% to about 3%, from about 0.8% to about 2%, from about 0.8% to about 1.5%, from about 0.9% to about 5%, from about 0.9% to about 4%, from about 0.9% to about 3%, from about 0.9% to about 2%, or from about 0.9% to about 1.5% by weight, relative to the total weight of the composition.

### Thickening Agents

In at least some embodiments, the compositions comprise one or more thickening agents. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, modified or unmodified starches, such as hydroxypropyl starch phosphate and/or potato starch, celluloses such as hydroxyethylcellulose and/or hydroxymethylcellulose, and guars such as hydroxypropyl guar.

In some embodiments, the thickening agent may be chosen from associative thickening polymers such as anionic associative polymers, amphoteric associative polymers, cationic associative polymers, nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may vary, but may be, for example, at least about 0.1%, such as at least about 0.25%, at least about 0.5%, at least about 0.75%, or at least about 1% by weight, based on the total weight of the composition. In some embodiments, the total amount of thickening agents may range from about 0.5% to about 5%, from about 0.5% to about 4%, from about 0.5% to about 3%, from about 0.5% to about 2%, from about 0.5% to about 1.5%, from about 0.6% to about 5%, from about 0.6% to about 4%, from about 0.6% to about 3%, from about 0.6% to about 2%, from about 0.6% to about 1.5%, from about 0.7% to about 5%, from about 0.7% to about 4%, from about 0.7% to about 3%, from about 0.7% to about 2%, from about 0.7% to about 1.5%, from about 0.8% to about 5%, from about 0.8% to about 4%, from about 0.8% to about 3%, from about 0.8% to about 2%, from about 0.8% to about 1.5%, from about 0.9% to about 5%, from about 0.9% to about 4%, from about 0.9% to about 3%, from about 0.9% to about 2%, or from about 0.9% to about 1.5% by weight, relative to the total weight of the composition.

### Carboxylic Acids

In various embodiments, compositions according to the disclosure optionally comprise at least one carboxylic acid. According to the disclosure, useful carboxylic acids include organic compounds that include, for example, one, two, three, or more acid functional groups and at least one carbon atom. Salts of carboxylic acids can also be used, and are expressly included in the term “carboxylic acid” unless stated otherwise.

These compounds typically have a molecular weight of less than about 500 g/mol, less than about 400 g/mol, or less than about 300 g/mol. Non-limiting examples of non-polymeric, mono, di, and/or tri-carboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, citric acid, isocitric acid, aconitric acid, propane-1,2,3-tricarboxylic acid, and benzene-1,3,5-tricarboxylic acid. In at least some embodiments, the carboxylic acid present in the composition comprises, consists essentially of, or consists of citric acid, maleic acid, malonic acid, or mixtures thereof.

If present, the compositions may comprise a total amount of carboxylic acids of at least about 0.1%, such as at least about 0.25%, at least about 0.5%, at least about 0.75%, or at least about 1% by weight, based on the total weight of the composition. In some embodiments, the total amount of carboxylic acids may range from about 0.5% to about 5%, from about 0.5% to about 4%, from about 0.5% to about 3%, from about 0.5% to about 2%, from about 0.5% to about 1.5%, from about 0.6% to about 5%, from about 0.6% to about 4%, from about 0.6% to about 3%, from about 0.6% to about 2%, from about 0.6% to about 1.5%, from about 0.7% to about 5%, from about 0.7% to about 4%, from about 0.7% to about 3%, from about 0.7% to about 2%, from about 0.7% to about 1.5%, from about 0.8% to about 5%, from about 0.8% to about 4%, from about 0.8% to about 3%, from about 0.8% to about 2%, from about 0.8% to about 1.5%, from about 0.9% to about 5%, from about 0.9% to about 4%, from about 0.9% to about 3%, from about 0.9% to about 2%, or from about 0.9% to about 1.5% by weight, relative to the total weight of the composition. For example, in some embodiments, the compositions comprise a total amount of carboxylic acids ranging from about 0.25% to about 1.75%, such as from about 0.35% to about 1.5%, from about 0.5% to about 1.25%, or from about 0.75% to about 1.25% by weight, relative to the total weight of the composition. In some exemplary embodiments, the compositions comprise at least one carboxylic acid chosen from citric acid, maleic acid, malonic acid, or mixtures of two or more thereof in an amount ranging from about 0.25% to about 1.75%, such as from about 0.35% to about 1.5%, from about 0.5% to about 1.25%, or from about 0.75% to about 1.25% by weight, relative to the total weight of the composition.

### Adjuvants

Optionally, compositions according to the disclosure may comprise one or more adjuvants. Non-limiting examples include preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the compositions (not for coloring the hair), sequestering agents, and the like.

The total amount of adjuvants, if present, typically ranges from about 0.01% to about 10% based on the total weight of the composition. For example, in some embodiments the individual amounts of each adjuvant or the total amount of adjuvants may range from about 0.1% to about 10%, about 0.1% to about 8%, about 0.1% to about 5%, about 0.1% to about 4%, about 0.1% to about 3%, about 0.1% to about 2%, about 1% to about 10%, about 1% to about 8%, about 1% to about 5%, about 1% to about 4%, about 1% to about 3%, or about 1% to about 2% by weight, based on the total weight of the composition.

According to some embodiments, the compositions of the present disclosure are free or substantially free of silicones and silicone-based compounds.

The pH of compositions according to the disclosure is generally less than about 7, for example the pH may range from about 2.5 to about 6, from about 3 to about 5, from about 3.5 to about 5, or from about 3.5 to about 4.5.

Compositions according to the disclosure are stable, meaning that no phase separation is seen for at least about 4 weeks, and in some embodiments at least about 8 weeks, at least about 12 weeks, at least about 24 weeks, at least about 6 months, at least about 12 months, at least about 2 years, at least about 3 years, or more.

II. Methods

The disclosure further relates to methods of treating, caring for, and/or conditioning keratin fibers, such as hair. The methods comprising applying a composition according to the disclosure onto the hair, optionally leaving the composition on the hair for a period of time (“rest period,” “resting period,” or “leave-in period”), and optionally rinsing the composition from the hair. The compositions according to the disclosure may thus be “leave-in” or “rinse-off” compositions.

In embodiments where the composition is a leave-in composition, the composition may be applied to the hair, which may be wet, damp, or dry. For example, in an exemplary embodiment, the hair may first be cleansed with a shampoo composition, rinsed, and then a composition according to the disclosure may be applied to the hair before the hair is dried, or before the hair is substantially dried, e.g. after excess water has been wrung from the hair and/or the hair has been towel-dried. After the leave-in composition is applied to the hair, the hair may be air dried or dried using a hair drier, and optionally styled. In such methods, the composition remains in the hair until the hair is next rinsed or washed in the ordinary course of personal grooming, e.g. for a period of time of about 12 hours, about 24 hours, about 36 hours, about 48 hours, etc.

In embodiments where the composition is a rinse-off composition, the composition may be applied to the hair, which may be wet, damp, or dry. For example, in an exemplary embodiment, the hair may first be cleansed with a shampoo composition, rinsed, and then a composition according to the disclosure may be applied to the hair before the hair is dried, or before the hair is substantially dried, e.g. after excess water has been wrung from the hair and/or the hair has been towel-dried. After the rinse-off composition is applied to the hair, the composition may be rinsed from the hair after an optional resting period, e.g. for a period of time lasting up to about 30 seconds, e.g. up to about one minute, up to about two minutes, up to about five minutes, up to about ten minutes, etc., such as from about 30 seconds to about 5 minutes, from about 1 minute to about 5 minutes, from about 2 minutes to about 10 minutes, etc. After the rinse-off composition according to the disclosure is rinsed from the hair, the hair may be air dried or dried using a hair drier, and optionally styled.

Surprisingly, keratin fibers that have been treated with compositions according to the disclosure exhibit both excellent conditioning benefits such as ease of detangling and softness, as well as excellent cosmetic properties such as excellent volume and smoothness, good static control, the ends have a sealed appearance, and the hair has a feeling of lightness or not being weighed down.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “citric acid and/or salts thereof” means “citric acid and salts thereof” as well as “citric acid or salts thereof,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials in an effective amount.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

It is to be understood that, with regard to salts of carboxylic acids described herein, it is intended to encompass the use of a salt of a carboxylic acid as an ingredient added to a composition according to the disclosure, or to the ion or salt of the carboxylic acid that forms when the carboxylic acid is used as an ingredient in a composition according to the disclosure.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “treat” (and its grammatical variations) refers to the application of the compositions of the present disclosure onto the surface of keratin fibers, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Compositions

Exemplary compositions 1A-1B according to the disclosure, and comparative compositions C1-C5, set forth in Table 1 were prepared.

TABLE 1

| | Inventive | | Comparative | | | | |
|---|---|---|---|---|---|---|---|
| | 1A | 1B | C1 | C2 | C3 | C4 | C5 |
| BEHENTRIMONIUM | 4.50 | 4.50 | 4.50 | 4.50 | — | 4.50 | 4.50 |
| CHLORIDE | | | | | | | |
| STEARAMIDOPROPYL | 0.50 | 0.50 | 0.50 | — | 0.50 | 0.50 | 0.50 |
| DIMETHYLAMINE | | | | | | | |
| GLYCERYL | 0.15 | 0.15 | 0.15 | 0.15 | 0.15 | — | 0.15 |
| STEARATE | | | | | | | |
| SORBITAN OLEATE | 0.04 | 0.04 | — | — | — | — | — |
| POLYSORBATE 20 | 0.80 | 0.80 | 0.80 | 0.80 | 0.80 | 0.80 | — |
| OCTYLDODECANOL | 1.00 | 1.00 | — | 1.00 | 1.00 | 1.00 | 1.00 |
| MODIFIED | 1.00 | 1.00 | 1.00 | 1.00 | 1.00 | 1.00 | 1.00 |
| POTATO STARCH | | | | | | | |
| POLYQUATERNIUM-37 | 1.00 | 0.50 | 1.00 | 1.00 | 1.00 | 1.00 | 1.00 |
| PROPYLENE GLYCOL | 0.70 | 0.70 | 0.70 | 0.70 | 0.70 | 0.70 | 0.70 |
| DICAPRYLATE/ | | | | | | | |
| DICAPRATE | | | | | | | |
| PPG-1 TRIDECETH-6 | 0.16 | 0.16 | 0.16 | 0.16 | 0.16 | 0.16 | 0.16 |
| CETEARYL ALCOHOL | 3.00 | 3.00 | 3.00 | 3.00 | 3.00 | 3.00 | 3.00 |
| CITRIC ACID | 1.00 | 1.00 | 1.00 | 1.00 | 1.00 | 1.00 | 1.00 |
| ADDITIVES (vitamins, | <2 | <2 | <2 | <2 | <2 | <2 | <2 |
| pH adjusters, | | | | | | | |
| preservatives, | | | | | | | |
| fragrance) | | | | | | | |
| SOLVENTS (water and | QS | QS | QS | QS | QS | QS | QS |
| non-aqueous solvent(s)) | | | | | | | |

### Example 2—Evaluation of Conditioning Benefits and Cosmetic Properties

The conditioning benefits and cosmetic properties imparted by compositions 1 A and C1-C5 according to Table 1 were evaluated.

### Example 2-1(A)

In order to evaluate the conditioning benefits and cosmetic properties provided by inventive composition 1A and comparative compositions C1-C5, two (2) experts with experience in performing such evaluations considered the following properties of six (6) separate swatches of bleached Caucasian hair that had been washed with a commercially-available shampoo, rinsed, had one of the tested compositions applied to the swatch which was left for a brief resting period, rinsed, and then dried: smoothness and ease of combing when wet, smoothness and ease of combing when dry, alignment of the hair fibers when dry, lightness of the hair fibers when dry, and seal of the ends of the hair. This type of evaluation is commonly performed to ascertain differences in conditioning and cosmetic benefits of compositions such as those described herein.

The experts agreed that inventive composition 1A, which comprises the synergistic combination of quaternary alkyl amines, tertiary alkyl amines, high HLB nonionic surfactants, low HLB nonionic surfactants, and branched alkyl alcohols, provided the most balanced combination of conditioning benefits and cosmetic properties to the hair of all of the compositions evaluated, followed in order by compositions C5, C₂, C₄, C₁, and C₃, where C₃ had the worst balanced combination of conditioning and cosmetic benefits.

### Example 2-1(B)

A bending study was then conducted on the hair swatches from Example 2-1(A). Hair swatches were fixed to a jig by the root end, and the bending angle, which is the degree the hair lays from the horizontal axis, was measured.

Each of the swatches treated with comparative compositions C2-C5 had a higher bending angle than that treated with inventive composition 1A, which indicates less volume was provided to the hair by compositions C2-C5 than by composition 1A. Although the swatch treated with comparative composition C₁ had a slightly lower bending angle than that treated with inventive composition 1A, meaning composition C1 provided slightly more volume to the hair than composition 1A, composition C₁ performed significantly worse than composition 1 A in Example 2-1(A).

In view of the results of Examples 2-1(A)-(B), the experts agreed that composition 1A provided the most balanced combination of conditioning benefits and cosmetic properties to the hair.

### Example 2-2

A panel of seven (7) professional hair stylists evaluated the conditioning and cosmetic properties imparted by composition 1A to the hair of four (4) individuals each. Each of the individuals was considered by the stylists to have fine hair (i.e. the hair fibers were not considered to be thick) that had been subjected to a chemical lightening treatment within the previous seven (7) days. Thus, a total of twenty-eight (28) evaluations of the performance of composition 1A on chemically-damaged hair were carried out.

All 28 individuals' hair was washed with the same commercially-available shampoo, the hair was rinsed, composition 1A was applied to the hair, and after a brief resting period, the hair was rinsed again, dried, and styled.

The results of the evaluations showed that the stylists unanimously agreed that hair treated with composition 1A was shiny and had a healthy appearance, felt soft and smooth, and had a lightweight rather than weighed-down appearance. These results are particularly surprising in view of the recent chemical treatment of the individuals' hair.

As demonstrated by the results of Examples 2-1 and 2-2, the synergistic combination of quaternary alkyl amines, tertiary alkyl amines, high HLB nonionic surfactants, low HLB nonionic surfactants, and branched alkyl alcohols, surprisingly provide the most balanced combination of conditioning benefits and cosmetic properties to the hair.

### Example 3—Additional Compositions

The following compositions according to the disclosure can be prepared, and are expected to perform similarly to composition 1 A to provide balanced conditioning benefits and cosmetic properties.

TABLE 2

| | 2A | 2B | 2C | 2D | 2E |
|---|---|---|---|---|---|
| CETRIMONIUM CHLORIDE | 4.50 | 2.50 | — | — | 2.50 |
| BEHENTRIMONIUM | — | — | 3.50 | 2.50 | 2.50 |
| METHOSULFATE | | | | | |
| STEARAMIDOPROPYL | 1.00 | 0.75 | — | — | 0.50 |
| DIMETHYLAMINE | | | | | |
| PALMITAMIDOPROPYL | — | — | 0.50 | 0.25 | 0.75 |
| DIETHYLAMINE | | | | | |
| GLYCERYL LAURATE | 0.75 | 0.20 | — | — | 0.15 |
| SORBITAN ISOSTEARATE | — | — | 0.50 | 0.25 | 0.15 |
| STEARETH-20 | 2.00 | 0.50 | — | — | 1.25 |
| LAURETH-23 | — | — | 1.25 | 1.00 | 1.00 |
| HEXYLDODECANOL | 1.00 | 0.50 | — | — | 0.50 |
| 2-METHYLDODECANOL | — | — | 1.25 | 1.50 | 0.50 |
| MODIFIED | 1.00 | 1.00 | 1.00 | 1.00 | 1.00 |
| POTATO STARCH | | | | | |
| POLYQUATERNIUM-37 | 1.00 | 1.00 | 1.00 | 1.00 | 1.00 |
| PROPYLENE GLYCOL | 0.70 | 0.70 | 0.70 | 0.70 | 0.70 |
| DICAPRYLATE/DICAPRATE | | | | | |
| PPG-1 TRIDECETH-6 | 0.16 | 0.16 | 0.16 | 0.16 | 0.16 |
| CETEARYL ALCOHOL | 3.00 | 3.00 | 3.00 | 3.00 | 3.00 |
| CITRIC ACID | 1.00 | 1.00 | 1.00 | 1.00 | 1.00 |
| ADDITIVES (vitamins, pH adjusters, | <2 | <2 | <2 | <2 | <2 |
| preservatives, fragrance) | | | | | |
| SOLVENTS (water and non- | QS | QS | QS | QS | QS |
| aqueous solvent(s)) | | | | | |
| Weight ratio of quaternary amine to | 4.50 | 3.33 | 7.00 | 10.0 | 4.00 |
| tertiary amine | | | | | |
| Weight ratio of high HLB to low HLB | 2.67 | 2.50 | 2.50 | 4.00 | 7.50 |
| nonionic surfactants | | | | | |

1. A hair treatment composition comprising:
(a) at least one quaternary alkyl amine;
(b) at least one tertiary alkyl amine;
(c) at least one nonionic surfactant with an HLB value of greater than or equal to about 15;
(d) at least one nonionic surfactant with a HLB value of less than or equal to about 5;
(e) at least one branched alkyl alcohol; and
(f) at least one solvent.

2. The hair treatment composition according to claim 1, wherein the at least one quaternary alkyl amine is chosen from quaternary ammonium salts of formula (I): 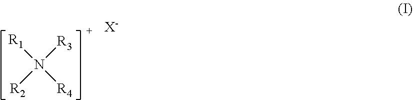
wherein:
R₁, R₂, R₃ and R₄ are independently chosen from saturated or unsaturated, linear or branched, aliphatic hydrocarbon radicals comprising from 1 to 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms; and
X⁻ is an anion.

3. The hair treatment composition according to claim 1, wherein the total amount of quaternary alkyl amine compounds present in the composition ranges from about 0.5% to about 10% by weight, based on the total weight of the composition.

4. The hair treatment composition according to claim 1, wherein the at least one tertiary alkyl amine is chosen from tertiary amido amine compounds.

5. The hair treatment composition according to claim 1, wherein the total amount of tertiary alkyl amine compounds ranges from about 0.01% to about 5% by weight, based on the total weight of the composition.

6. The hair treatment composition according to claim 1, wherein:
the at least one quaternary alkyl amine comprises at least one compound chosen from behentrimonium chloride, cetrimonium chloride, behentrimonium methosulfate, or mixtures thereof; and/or
the at least one tertiary alkyl amine compound comprises stearamidopropyldimethylamine.

7. The hair treatment composition according to claim 1, having a weight ratio of the total amount of quaternary alkyl amines to the total amount of tertiary alkyl amines ranging from about 1 to about 12.

8. The hair treatment composition according to claim 1, wherein the total amount of nonionic surfactants with an HLB value of greater than or equal to about 15 in the composition ranges from about 0.01% to about 5% by weight, based on the total weight of the composition.

9. The hair treatment composition according to claim 1, wherein the total amount of nonionic surfactants with an HLB value of lower than or equal to about 5 in the composition ranges from about 0.01% to about 5% by weight, based on the total weight of the composition.

10. The hair treatment composition according to claim 1, having a weight ratio of the total amount of nonionic surfactants with an HLB value of greater than or equal to about 15 to the total amount of nonionic surfactants with an HLB value of lower than or equal to about 5 ranging from about 1 to about 10.

11. The hair treatment composition according to claim 1, wherein the at least one branched alkyl alcohol is chosen from branched, saturated alcohols containing at least 8 carbon atoms.

12. The hair treatment composition according to claim 1, wherein the total amount of branched alkyl alcohols in the composition ranges from about 0.01% to about 6% by weight, based on the total weight of the composition.

13. The hair treatment composition according to claim 1, further comprising at least one additional component chosen from cationic polymers, carboxylic acids, thickening agents, or combinations thereof.

14. The hair treatment composition according to claim 1, wherein the composition is substantially free of silicones.

15. The hair treatment composition according to claim 1, having a pH ranging from about 2.5 to about 6.

16. A hair treatment composition comprising:
(a) from about 1% to about 6% of quaternary alkyl amine(s) chosen from compounds of formula (I): 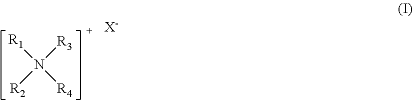
wherein:
R₁, R₂, R₃ and R₄ are independently chosen from saturated or unsaturated, linear or branched, aliphatic hydrocarbon radicals comprising from 1 to 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms; and
X⁻ is an anion;
(b) from about 0.1% to about 3% of tertiary amido amine(s);
(c) from about 0.1% to about 3% of nonionic surfactant(s) with an HLB value of greater than or equal to about 15;
(d) from about 0.01% to about 2% of nonionic surfactant(s) with a HLB value of less than or equal to about 5;
(e) from about 0.1% to about 3% of branched alkyl alcohol(s); and
(f) water,
wherein the composition is substantially free of silicones.

17. The hair treatment composition according to claim 16, having a weight ratio of the total amount of quaternary alkyl amines of formula (I) to the total amount of tertiary amido amines ranging from about 3 to about 10.

18. The hair treatment composition according to claim 16, having a weight ratio of the total amount of nonionic surfactants with an HLB value of greater than or equal to about 15 to the total amount of nonionic surfactants with an HLB value of lower than or equal to about 5 ranging from about 3 to about 7.

19. A method of treating hair, comprising applying to the hair a composition comprising:
(a) at least one quaternary alkyl amine;
(b) at least one tertiary alkyl amine;
(c) at least one nonionic surfactant with an HLB value of greater than or equal to about 15;
(d) at least one nonionic surfactant with a HLB value of less than or equal to about 5;
(e) at least one branched alkyl alcohol; and
(f) at least one solvent.

20. The method according to claim 19, wherein the composition comprises:
(a) from about 1% to about 6% of quaternary alkyl amine(s) chosen from compounds of formula (I): 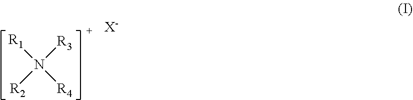
wherein:
R₁, R₂, R₃ and R₄ are independently chosen from saturated or unsaturated, linear or branched, aliphatic hydrocarbon radicals comprising from 1 to 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms; and
X⁻ is an anion;
(b) from about 0.1% to about 3% of tertiary amido amine(s);
(c) from about 0.1% to about 3% of nonionic surfactant(s) with an HLB value of greater than or equal to about 15;
(d) from about 0.01% to about 2% of nonionic surfactant(s) with a HLB value of less than or equal to about 5;
(e) from about 0.1% to about 3% of branched alkyl alcohol(s); and
(f) water.
